(19) United States
(12) Patent Application Publication
(10) Pub. No.: US 2009/0318967 A1
(43) Pub. Date: Dec. 24, 2009

(54) Spacer for use in a surgical operation for spinous process of spine

(76) Inventors: Dong Myung Jeon, Draper, UT (US); Hee Jin Yang, Seongnam-si (KR)

Correspondence Address: MORRISS OBRYANT COMPAGNI, P.C., 734 EAST 200 SOUTH, SALT LAKE CITY, UT, 84102, US

(21) Appl. No.: 12/460,726

(22) Filed: Jul. 23, 2009

Related U.S. Application Data
(63) Continuation of application No. PCT/KR08/00429, filed on Jan. 23, 2008.

(30) Foreign Application Priority Data
Jan. 23, 2007 (KR) 10-2007-0007206
Jan. 23, 2008 (KR) 10-2008-0007097
Jul. 31, 2008 (WO) WO 2008/091112

(51) Int. Cl. A61B 17/70; A61B 17/88

(52) U.S. Cl. 606/249; 606/279

(57) ABSTRACT

A spacer used for maintaining an interval between spinous processes of spine, capable of preventing unnecessary damage to vertebras, simplifying the surgical operation, and reducing the time taken for the surgical operation by implementing a new vertebra fastening means for strongly maintaining a state of extending an interval between spinous processes by using a shape memory material which can be restored by body temperature. In addition, such a spacer may be capable of easily insertion in a lateral direction, thereby reducing a cut portion for the surgical operation and avoiding unnecessary cutting of a ligament fastening the spinous processes. In addition, there is provided a spacer for use in a surgical operation for spinous processes of spine, capable of enabling the spine to move according to patient's movement by providing an elastic vertebra fastening means, so that the patient can move smoothly after the surgical operation.

### TECHNICAL FIELD

The present invention relates to a spacer inserted between spinous processes of spine to fasten the vertebras, and more particularly to, a spacer for use in a surgical operation for spinous processes of spine, capable of maintaining a constant interval between spinous processes and stably fastening vertebras by using characteristics of a shape memory material and capable of inserting the spacer in a lateral direction of the spinous process during a surgical operation to minimize a cut portion.

### BACKGROUND ART

In general, if a portion of spine or a vertebra is impaired, the patient cannot move in the state. Although a patient having non-serious impairment can move, an impaired portion of the spine is contacted or pressed by an adjacent portion, so that the patient suffers a pain. In addition, although the impaired portion is cured, a long time is taken for the recovery.

Therefore, in case of the patient whose spine is partially impaired or damaged, a surgical operation of inserting an artificial assistant material for supporting the adjacent portion of the impaired vertebra is performed so that the impaired portion of the vertebra is not pressed by the adjacent portion of the vertebra.

In this case, a vertebra fastening mechanism is used. The vertebra fastening mechanism is constructed with screw nails which are inserted at upper and lower sides of the impaired portion of the vertebra to serve as a fastening member and rods which are connected with the screw nails to serve as a supporting member.

Now, a surgical operation using the vertebra fastening mechanism 20 will be described.

Firstly, as shown in FIG. 6, the screw nails 30 are vertically screwed to to-be-connected vertebras 100. Next, rods 40 are inserted to heads of the screw nails 30 and rods are fixed to the heads. Next, fixing bolts are engaged with the heads of the screw nails 30 by using a tool, so that the rods 40 can be pressed. As a result, a firmly-fixed state of the rods 40 connecting the vertebras can be maintained.

However, in the surgical operation using the vertebra fastening mechanism, it is difficult to accurately engage the screw nails and the rods, and a long time is taken for performing the surgical operation. In addition, since a sufficient engagement force is not provided, a minute movement of the rods may cause disengagement of the engagement state.

In the existing surgical operation using the vertebra fastening mechanism, a damaged portion or a malformed portion (scoliosis or kyphosis) of a vertebra disk is cut, or deformed array of vertebras are aligned. In this state, drilling is performed at both sides of the corresponding portion, screw nails are inserted, and the screw nails are connected with rods. In this case, the surgical operation is very complicated, and a long time is taken for performing the surgical operation. In addition, a fastening force for connecting the screw nails with the rods cannot be easily adjusted, and a lot of damages to the bone may occur at the surgical operation.

Particularly, in a case where the impaired vertebra is fastened by using rods, since the rod has no elasticity, the vertebra fastened by the rods cannot be moved, a weight is concentrated on the upper and lower vertebras of the fastened vertebra, so that a patient bending the waist has unpleasantness or pain.

These aforementioned problems become impediments to spine surgery requiring for speedy and accurate surgical operations, so that there is a need to solve the problems.

### DISCLOSURE OF INVENTION

1. Technical Problem

In order to solve the aforementioned problems, the present invention provides a spacer for use in a surgical operation for spinous processes of spine, capable of preventing unnecessary damage to vertebras, simplifying the surgical operation, and reducing a time taken for the surgical operation by implementing a new vertebra fastening means of strongly maintaining a state of extending an interval between the spinous processes of spine by using a shape memory material which can be restored by a body temperature.

In addition, the present invention also provides a spacer for use in a surgical operation for spinous processes of spine, capable of easily inserting the vertebra fastening means in a lateral direction, thereby reducing a cut portion for the surgical operation and avoiding unnecessary cutting of a ligament of fastening the spinous processes.

In addition, the present invention also provides a spacer for use in a surgical operation for spinous processes of spine, capable of enabling a vertebra to move according to patient's movement by providing an elastic vertebra fastening means, so that the patient can move smoothly after the surgical operation.

2. Technical Solution

According to an aspect of the present invention, there is provided a spacer inserted between spinous processes of spine, comprising: a pair of receiving members, each of which is made of a shape memory material and covers and fastens a pair of adjacent spinous processes; and a supporting member which is made of a shape memory material and connected to a pair of the receiving members, wherein the supporting member is restored by a body temperature in a direction for separating the receiving members to exert an extending force for extending and maintaining an interval between the spinous processes.

In the above aspect of the present invention, at least one side of each of the receiving member may have a horizontal portion having a straight shape which can be vertically restored.

Preferably, in a case where only one side of each of the receiving members is formed to be a horizontal portion, the receiving member is constructed with a concave structure where a vertically-restored horizontal portion together with the other vertical portion can be inserted into the spinous process.

In addition, the supporting member may be formed to have a U-shaped structure where an opening portion is connected to one side of each of the receiving members, so that the supporting member can be restored from a folded U-shaped structure where end portions of the opening portion connected to the receiving members are almost close to each other to a normally-unfolded U-shaped structure.

In addition, the supporting member is formed to have a W-shaped structure where an opening portion is connected to one side of each of the receiving members, so that the supporting member can be restored from a folded W-shaped structure where end portions of the opening portion connected to the receiving members are almost close to each other to a normally-unfolded W-shaped structure.

Preferably, a connection portion between the receiving member and the supporting member is formed to be a notch for facilitating deformation of the receiving member.

### BRIEF DESCRIPTION OF THE DRAWINGS

FIG. 1 is a perspective view illustrating a before-restoring state of a spacer for use in a surgical operation for spinous processes of spine according to an embodiment of the present invention.

FIG. 2 is a perspective view illustrating an after-restoring state of the spacer for use in a surgical operation for spinous processes of spine according to the embodiment of the present invention.

FIG. 3 is a front view illustrating before-restoring and after-restoring states of the spacer for use in a surgical operation for spinous processes of spine according to the embodiment of the present invention.

FIG. 4 is a side view illustrating before-restoring and after-restoring states of the spacer for use in a surgical operation for spinous processes of spine according to the embodiment of the present invention.

FIG. 5 is a front view illustrating a surgical state of the spacer for use in a surgical operation for spinous processes of spine according to the embodiment of the present invention.

FIG. 6 is a schematic view illustrating a vertebra surgery method using a conventional vertebra fastening mechanism.

FIG. 7 is a perspective view illustrating an after-restoring state of a spacer for use in a surgical operation for spinous processes of spine according to another embodiment of the present invention.

FIG. 8 is a perspective view illustrating before-restoring and after-restoring states of the spacer for use in a surgical operation for spinous processes of spine according to another embodiment of the present invention, shown in FIG. 7.

### BEST MODE FOR CARRYING OUT THE INVENTION

Hereinafter, embodiments of the present invention will be described in detail with reference to the accompanying drawings.

In the specification and claims, terms are not limited to general or lexicographic meanings. Since an inventor can suitably define terms for the best description of the present invention, the terms should be understood to be interpreted as meanings and concepts suitable for the technical idea of the present invention.

While embodiments and constructions disclosed in the specification and the drawings are exemplary ones that do not cover all the scope of the present invention, it should be understood that the present invention can be implemented with various modifications and equivalents on the filling date of the present invention.

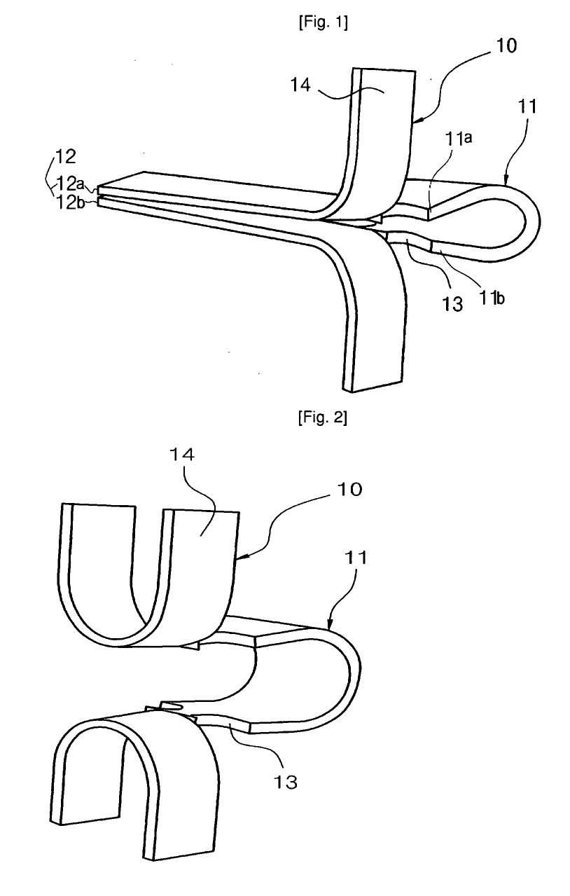

FIGS. 1 and 2 are perspective views illustrating before-restoring and after-restoring states of a spacer for use in a surgical operation for spinous processes of spine according to an embodiment of the present invention.

As shown in FIGS. 1 and 2, the spacer for use in a surgical operation for the spinous processes according to the present invention is made of a shape memory material. The spacer includes a pair of receiving members 10 for fastening upper and lower adjacent spinous processes of the vertebra and a supporting member 10 for exerting upward and downward extending forces to the receiving members 10 to support the receiving members 10.

The upper and lower receiving members 10 have upper and lower receiving surfaces for receiving upper and lower spinous processes, respectively. Each of the receiving members 10 has a concave structure for covering and fastening the spinous process.

The concave structure is formed during a restoring process of the shape memory material after the surgical operation. The concave structure has a function of preventing the spacer from being separated and moved from the spinous processes after the surgical operation.

The concave structure is not limited to a substantially U-shaped structure shown in the figures, but a V-shaped structure and any other structures capable of receiving the spinous processes can be employed.

Before the restoring process of the receiving members 10, each of the receiving members 10 has a horizontal portion 12 and a vertical portion 14.

During the restoring process, the horizontal portion 12 of the receiving member 10 is folded in the vertical direction due a body temperature after the surgical operation. Therefore, the vertically-restored horizontal portion 12 and the vertical portion 14 constitute the concave structure (that is, the substantially U-shaped structure) for stably covering and fastening the spinous process.

In the spacer according to the present invention, due to the horizontal portion 12, the spacer can be easily inserted between the spinous processes in the lateral direction of the spinous processes. Therefore, the cut portion for the surgical operation can be reduced, and a ligament needs not to be cut

In addition, the supporting member 11 is connected integrally to the upper and lower receiving members 10. After the surgical operation, the supporting member is restored in up and down directions due to the body temperature to separate the upper and lower receiving members 10 from each other. That is, the supporting member 11 exerts extending forces to the upper and lower adjacent spinous processes so as to extend and maintain the interval between the adjacent spinous processes.

The supporting member 11 is connected to side portions of the upper and lower receiving members 10. More specifically, upper and lower opening ends 11a and 11b of the supporting members 11 are connected to centers of side portions (width-directional end portions) of the upper and lower receiving members 10, respectively.

In this manner, since an opening portion 11c of the supporting member 11 is connected to the side portions of the receiving members 10, a contracting force of the supporting member 11 can be increased, so that a total height of the spacer before insertion between the spinous processes can be further reduced. That is, the supporting member 11 connected to the centers of the side portions of the receiving members 10 can be contracted so that the upper and lower ends 11a and 11b of the supporting member 11 are almost close to each other. Therefore, the upper and lower horizontal portions 12a and 12b inserted between the adjacent spinous processes in the lateral direction are also almost close to each other. Accordingly, the cut portion for the surgical operation can be reduced down to about ⅓ times the cut portion for the surgical operation using a conventional product.

In addition, the upper horizontal portion 12a and the lower horizontal portion 12b may be further deformed downward and upward to be close to each other, respectively. In order to more easily deform the horizontal portions, notches 13 are formed in the connection portions between the supporting member 11 and the receiving members 10. Due to the notches 13, the upper and lower horizontal portions 12a and 12b can be easily deformed to be slanted (not to be parallel to each other) so as to reduce the separation therbetween toward the ends of the upper and lower horizontal portions 12a and 12b. Accordingly, the cut portion for the surgical operation can be further reduced.

The supporting member 11 is made of a shape memory material so as to restore the spacer inserted between the spinous processes into an original state by using the body temperature.

Preferably, the receiving members 10 and the supporting member 11 are formed integrally, and the receiving members 10 as well as the supporting member 11 are made of the shape memory material.

Now, constructions of the spacer where the receiving members 10 and the supporting member 11 are integrally formed by using the shape memory material will be described.

As an example of the shape memory material, a shape memory alloy containing titanium-nickel as a main ingredient may be used. The present invention is not limited to the shape memory alloy, but a shape memory polymer, a shape memory ceramic, and any other shape memory material known to the ordinarily skilled in the art may be used. Hereinafter, the shape memory alloy will be exemplified as the shape memory material.

A shape memory process for the shape memory alloy is performed as follows. Firstly, a wire or a strip of the shape memory alloy is formed in a shape which is to be maintained after the surgical operation. Next, in a state that the wire or the strip is fixed so as not to be deformed during the following thermal treatment process, the shape memory thermal treatment process is performed at a temperature of 300 to 900° C. for 10 minutes.

In an alternative example, the shape memory process may be performed as follows. Firstly, a plate having predetermined length and width is formed by using a cutting machine in a shape which is to be maintained after the surgical operation. Next, the shape memory thermal treatment process is performed at a temperature of 300 to 900° C. for 10 minutes.

In another alternative example of the shape memory process, in a state that a wire, a strip, or a plate is confined by a jig having a shape corresponding to the shape which is to be maintained after the surgical operation, the shape memory thermal treatment process is performed at a temperature of 300 to 900° C. for 10 minutes.

Due to the shape memory process, the supporting member 11 of the spacer according to the embodiment of the present invention has a structure which can be restored from a semi-elliptic shape to a semi-circular shape. More specifically, due to the shape memory process, the supporting member 11 has a structure which can be restored from a folded U-shape (where ends of the supporting member 11 is deformed to be almost close to each other) to an unfolded U-shape. The horizontal portion 12 of each receiving member 10 can be restored from a shape where the horizontal portion extends in the horizontal direction to a shape where the horizontal portion is erected in the vertical direction.

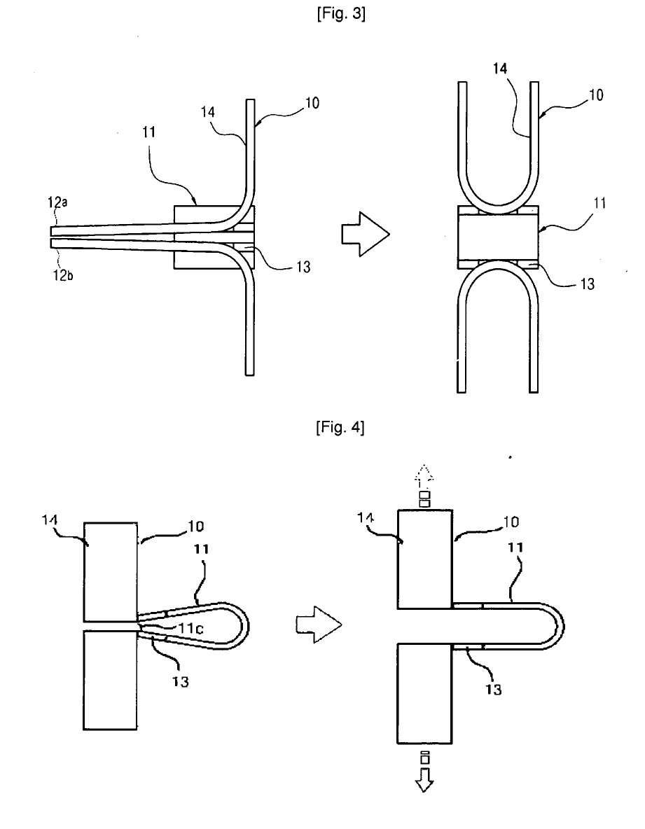

As shown in FIG. 3, the spacer is surgically operation in a lateral direction of the spinous process by using horizontal portions 12a and 12b in a Martensite state of which temperature is less than about a transition temperature of 25° C. More specifically, the spacer is surgically operation in a state that the supporting member 11 is contracted to be straight, so that the supporting member has a height lower than an interval between the spinous processes adjacent to the operated portion.

In about one to two minutes after the surgical operation, a temperature of the spacer is gradually increased due to the body temperature. When the temperature of the spacer exceeds the transition temperature, the supporting member 11 and the receiving members 10 of the spacer are restored into a before-contraction state. Particularly, each of the horizontal portions 12a and 12b of the receiving members 10 is restored in the vertical direction, so that the restored horizontal portion and the vertical portion 14 constitutes a U-shaped structure for covering and fastening the spinous process of the spine.

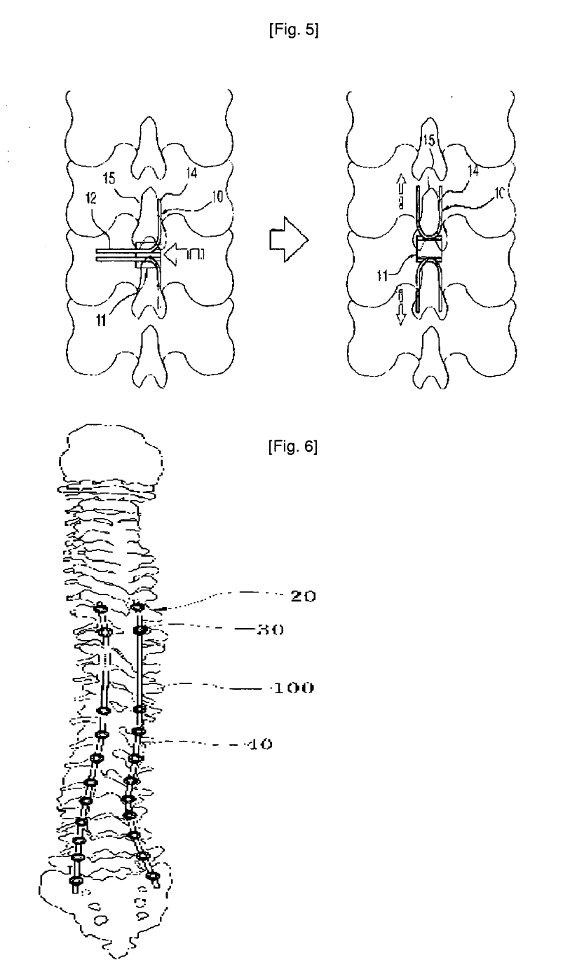

FIG. 5 is a view illustrating a surgical state where the spacer is inserted and restored between the spinous processes.

As shown in FIG. 5, since the supporting member 11 and the receiving members 10 of the spacer are restored due to the body temperature, the interval between the spinous processes 15 are extended and maintained. In addition, since the spinous processes 15 are received into the receiving members 10 to be covered with the concave structures of the receiving members 10, the spacer can be prevented from being separated from the spinous processes 15 during the movement of the spine.

Particularly, according to the spacer of the present invention, since the before-deformation receiving members 10 have horizontal portions 12 having a straight shape, the spacer can be inserted between the spinous processes 15 in the lateral direction, so that it is possible to stably and efficiently perform the surgical operation associated with the insertion of the spacer between the spinous processes.

In addition, unlike a conventional method where screw nails are connected by using rods, in the present invention, since the spacer is inserted between the spinous processes so as to fasten the vertebra, the vertebra can be slightly moved due to the elasticity of the spacer, so that it is possible to minimize patient's unpleasantness during the patient's movement.

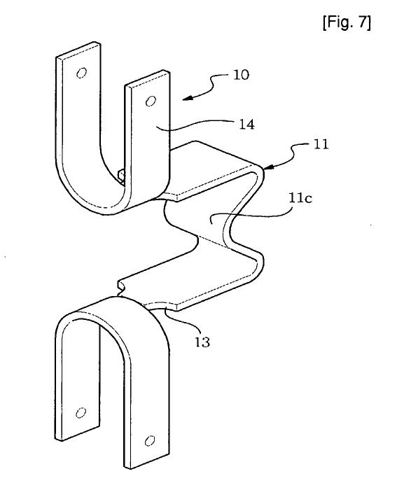

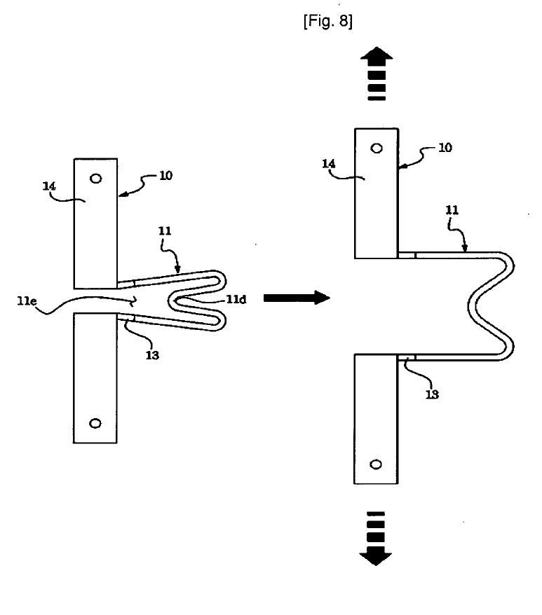

In the above embodiment of the present invention, the supporting member 11 is formed to have a horizontally U-shaped structure. In an alternative embodiment of the present invention, as shown in FIGS. 7 and 8, the supporting member 11 may be formed to have a horizontally W-shaped structure where upper and lower end portions of an opening portion I le is almost close to each other. In addition, since a shape memory process is performed on the supporting member, the horizontally W-shaped structure can be restored to a normally-unfolded W-shaped structure.

In this manner, in comparison with the U-shaped structure, the horizontally W-shaped structure of the supporting member is further provided with an internally curved buffering portion lid. Therefore, after the surgical operation, it is possible to maintain a state of extending the interval between the adjacent spinous processes strongly for a long time.

### INDUSTRIAL APPLICABILITY

According to the present invention, since damage to spine during a surgical operation can be greatly reduced, it is possible to precipitate recovering a function of the spine. In addition, since the spacer disposed to a surgical portion can be automatically extended to be fastened between the spinous processes by a body temperature in about one or two minutes, it is possible to simplify the surgical operation and to reduce a time taken for the surgical operation in comparison with a conventional method.

In addition, due to a concave structure (U-shaped structure) of a receiving member that is fastened to cover the spinous process, since the spacer is prevented from being separated from the spinous process during leftward, rightward, forward, and backward movements of the spine, it is possible to stably maintain a surgical state.

In addition, according to the present invention, since the receiving member that is deformed in a straight shape can be easily inserted, the cut portion for the surgical operation can be reduced down to about ⅓ times the cut portion for the surgical operation using a conventional product. Since the receiving member can be inserted in the lateral direction of the spinous process, unnecessary cutting of a ligament of fastening the spinous process can be avoided, so that it is possible to maintain a stability. Particularly, since the spacer having a large size can be contracted, the spacer can be used for a surgical operation using an endoscopic tube or a small-diameter tube, so that it is possible to greatly reduce a size of the cut portion in comparison with a conventional method.

In addition, since the spacer according to the present invention has an elasticity, the spine can be slightly moved during a patient's movement, so that it is possible to prevent a portion other than the surgical portion from being greatly moved.

1-6. (canceled)

7. A spacer for insertion between the spinous processes of adjacent vertebrae, comprising:
a first receiving member constructed of a shape memory material, the first receiving member formed as a member with a first end and a second end, which restores to a concave shape for engaging a first spinous process on a first vertebra by restoration of shape memory;
a second receiving member constructed of the shape memory material, the second receiving member formed as a member with a third end and a fourth end, which restores to a concave shape for engaging a second spinous process on a second vertebra adjacent to the first vertebra by restoration of shape memory; and
a supporting member constructed of shape memory material which is connected to the first receiving member and the second receiving member.

8. The spacer of claim 7, wherein the first receiving member constructed of a shape memory material, the first receiving member formed as a member with a first end and a second end, which restores to a concave shape for engaging a first spinous process on a first vertebra by restoration of shape memory comprises the first receiving member formed as a member with a first section which extends from the first end to a first bend and a second section which extends from the first bend to the second end, with the first section and second section extending generally perpendicular to one another from the first bend in an unrestored shape, which restores to a concave shape for engaging a first spinous process on a first vertebra by movement of the first section and second section by restoration of shape memory.

9. The spacer of claim 7, wherein the first receiving member is constructed of a titanium-nickel alloy shape memory material.

10. The spacer of claim 7, wherein the first receiving member is constructed of a shape memory material which restores to a remembered shape by exposure to body temperature.

11. The spacer of claim 7, wherein the first receiving member constructed of a shape memory material, the first receiving member formed as a member with a first end and a second end, which restores to a concave shape for engaging a first spinous process on a first vertebra by restoration of shape memory comprises a member with a first end and a second end, which restores to a generally U-shaped configuration.

12. The spacer of claim 7, wherein the second receiving member constructed of the shape memory material, the second receiving member formed as a member with a third end and a fourth end, which restores to a concave shape for engaging a second spinous process on a second vertebra adjacent to the first vertebra by restoration of shape memory comprises the second receiving member formed as a member with a third section which extends from the third end to a second bend and a fourth section which extends from the second bend to the fourth end, with the third section and fourth section extending generally perpendicular to one another from the second bend in an unrestored shape, which restores to a concave shape for engaging a second spinous process on a second vertebra adjacent to the first vertebra by movement of the third section and fourth section by restoration of shape memory.

13. The spacer of claim 7, wherein the supporting member constructed of shape memory material which is connected to the first receiving member and the second receiving member comprises a member which is connected at either end to a midpoint of the concave shape of the respective receiving member in a restored state.

14. The spacer of claim 7, wherein the supporting member constructed of shape memory material which is connected to the first receiving member and the second receiving member comprises a member which is in a retracted position in an unrestored state, which restores to an extended position by restoration of shape memory separating the first receiving member from the second receiving member.

15. The spacer of claim 14, wherein separating the first receiving member from the second receiving member exerts an extending force for extending and maintaining an interval between the first spinous process and second spinous process.

16. The spacer of claim 7, wherein the supporting member constructed of shape memory material which is connected to the first receiving member and the second receiving member comprises a member which, upon restoration, has a generally U-shaped configuration or a generally W-shaped configuration.

17. The spacer of claim 7, wherein the first receiving member, the supporting member, and the second receiving member are integrally constructed from the same shape memory material.

18. A method for inserting a spacer between the spinous processes of adjacent vertebrae, comprising:
converting a spacer constructed of a shape memory material into a nonrestored state, the spacer comprising
a first receiving member constructed of a shape memory material, the first receiving member formed as a member with a first section which extends from a first end to a first bend and a second section which extends from the first bend to a second end, with the first section and second section extending generally perpendicular to one another from the first bend in an unrestored shape, which restores to a concave shape for engaging a first spinous process on a first vertebra by movement of the first section and second section by restoration of shape memory,
a second receiving member constructed of the shape memory material, the second receiving member formed as a member with a third section which extends from a third end to a second bend and a fourth section which extends from the second bend to a fourth end, with the third section and fourth section extending generally perpendicular to one another from the second bend in an unrestored shape, which restores to a concave shape for engaging a second spinous process on a second vertebra adjacent to the first vertebra by movement of the third section and fourth section by restoration of shape memory, and
a supporting member constructed of shape memory material which is connected to the first receiving member and the second receiving member;
inserting the spacer into an interspinous space between the first spinous process on the first vertebra and the second spinous process on the second vertebra by laterally placing the spacer into the interspinous space by sliding the first section of the first receiving member and the third section of the second receiving member past the first and second spinous processes; and
restoring the memory shape material of the spacer to a restored state to engage the first spinous process in the first receiving member and the second spinous process in the second receiving member.

19. The method of claim 18, wherein converting a spacer constructed of a shape memory material into a nonrestored state comprises converting a spacer constructed of a titanium-nickel alloy shape memory material.

20. The method of claim 18, wherein restoring the memory shape material of the spacer to a restored state to engage the first spinous process in the first receiving member and the second spinous process in the second receiving member comprises exposing the spacer to body temperature.

21. The method of claim 18, wherein converting a spacer constructed of a shape memory material into a nonrestored state, the spacer comprising a first receiving member constructed of a shape memory material, the first receiving member formed as a member with a first section which extends from a first end to a first bend and a second section which extends from the first bend to a second end, with the first section and second section extending generally perpendicular to one another from the first bend in an unrestored shape, which restores to a concave shape for engaging a first spinous process on a first vertebra by movement of the first section and second section by restoration of shape memory comprises converting a spacer comprising a first receiving member which restores to a generally U-shaped configuration.

22. The method of claim 18, wherein converting a spacer constructed of a shape memory material into a nonrestored state which comprises a supporting member constructed of shape memory material which is connected to the first receiving member and the second receiving member comprises converting a spacer comprising a supporting member which is connected at either end to a midpoint of the concave shape of the respective receiving member in a restored state.

23. The method of claim 18, wherein converting a spacer constructed of a shape memory material into a nonrestored state which comprises a supporting member constructed of shape memory material which is connected to the first receiving member and the second receiving member comprises converting a spacer comprising a support member which is in a retracted position in an unrestored state, which restores to an extended position by restoration of shape memory separating the first receiving member from the second receiving member.

24. The method of claim 23, wherein restoring the memory shape material of the spacer to a restored state to engage the first spinous process in the first receiving member and the second spinous process in the second receiving member further comprises separating the first receiving member from the second receiving member by restoring the support member to an extended position thereby exerting an extending force for extending and maintaining an interval between the first spinous process and second spinous process.

25. The method of claim 18, wherein converting a spacer constructed of a shape memory material into a nonrestored state which comprises a supporting member constructed of shape memory material which is connected to the first receiving member and the second receiving member comprises converting a spacer comprising a supporting member which, upon restoration, has a generally U-shaped configuration or a generally W-shaped configuration.

26. The method of claim 18, wherein converting a spacer constructed of a shape memory material into a nonrestored state comprises converting a spacer which is integrally constructed from a single piece of shape memory material.
